(19) United States
(12) Patent Application Publication
(10) Pub. No.: US 2010/0256529 A1
(43) Pub. Date: Oct. 7, 2010

(54) Prenatal Listening Device

(75) Inventors: Elizabeth Grasing, Ardmore, PA (US); Joshua Clapper, Downingtown, PA (US); Craig Desrosiers, Spring City, PA (US)

Correspondence Address: Lempia Braidwood Graco, One North LaSalle Street Suite 3150, Chicago, IL, 60602, US

(73) Assignee: Graco Children's Products Inc., Exton, PA (US)

(21) Appl. No.: 12/493,727

(22) Filed: Jun. 29, 2009

Related U.S. Application Data
(60) Provisional application No. 61/167,393, filed on Apr. 7, 2009.

(51) Int. Cl. A61B 7/00

(52) U.S. Cl. 600/586

(57) ABSTRACT

A prenatal listening device includes first and second handheld units. The first handheld unit includes a cup-shaped body that defines an acoustic cavity. The first handheld unit further includes a microphone mounted to the body in the acoustic cavity to capture fetal sounds collected by the acoustic cavity. The second handheld unit is spaced from and communicatively coupled to the first handheld unit. The second handheld unit includes a controller to process signals from the microphone indicative of the captured fetal sounds, a housing to enclose the controller, and an audio output to reproduce the captured fetal sounds.

### CROSS-REFERENCE TO RELATED APPLICATION

This application claims the benefit of U.S. provisional application entitled “Prenatal Heart Monitor,” filed Apr. 7, 2009, and having Ser. No. 61/167,393, the entire disclosure of which is hereby expressly incorporated by reference.

### BACKGROUND OF THE DISCLOSURE

1. Field of the Disclosure

The present disclosure is generally directed to devices used to capture and reproduce sounds from the womb, and more particularly to fetal stethoscopes and other devices used for prenatal heart monitoring or listening.

2. Description of Related Art

There are several reasons for monitoring or listening to a fetal heartbeat. During delivery, the heartbeat is often continuously monitored to detect signs of stress. The heartbeat is also frequently monitored in high-risk pregnancies. Parents may also wish to listen to the heartbeat outside of these circumstances for a variety of non-medical reasons.

Only a subset of the commercially available devices and techniques for listening to fetal heartbeats are suitable for home use. For instance, electronic techniques involving electrode arrangements are best handled by a medical professional even when external (i.e., when placed on the skin of the abdomen). Electronic techniques may also be undesirably complex due to the machines used for generating and displaying the waveforms representative of the electrical activity in the womb.

Other methods of capturing sounds from the womb involve ultrasound-based methods. For example, some methods utilize devices that rely on the Doppler Effect. These handheld devices emit a radar wave into the womb to detect the phase shift from movement therein. While parents may be capable of finding a fetal heartbeat with a Doppler device, these devices are undesirably expensive and invasive.

Fetoscopes are stethoscopes designed specifically for listening to sounds within the womb. These stethoscopes are also known as Pinard's stethoscopes or pinards. The technique is noninvasive, and simple to use for those individuals trained in the nursing or other medical professions. Unfortunately, it is often difficult for someone unfamiliar with a fetoscope to use it to locate a heartbeat. Furthermore, a mother is unable to use a fetoscope on herself, inasmuch as fetoscopes are designed to be braced against the listener's forehead.

There are several commercially available listening devices targeted to parents for home use. For example, a device marketed under the Summer Infant brand uses a piezoelectric sensor as a microphone. A prenatal heart listener has also been sold under the BebeSounds brand. Unfortunately, these products are often considered by parents to provide inadequate performance, particularly at points early in the pregnancy.

### BRIEF DESCRIPTION OF THE DRAWINGS

Objects, features, and advantages of the present invention will become apparent upon reading the following description in conjunction with the drawing figures, in which like reference numerals identify like elements in the figures, and in which:

FIG. 1 is an exploded, perspective view of an exemplary electronic prenatal heart listener with separate main and auxiliary units constructed in accordance with several aspects of the disclosure;

FIG. 2 is a partial, perspective view of the electronic prenatal heart listener of FIG. 1 to depict the main unit and the auxiliary unit arranged and coupled in a storage configuration in accordance with one aspect of the disclosure;

FIGS. 3A and 3B are front and side perspectives of one embodiment of the auxiliary unit of the electronic prenatal heart listener of FIGS. 1 and 2 to depict a squared bowl-shaped body having an elongated-knob handle in accordance with one aspect of the disclosure;

FIGS. 4A and 4B are bottom view of alternative embodiments of the auxiliary unit of the electronic prenatal heart listener of FIGS. 1 and 2 to depict spherical and non-spherical acoustic or sound collection cavities configured in accordance with several aspects of the disclosure;

FIG. 5 is a partial, schematic, side view of one embodiment of the auxiliary unit of the electronic prenatal heart listener of FIG. 1 with a two-piece body to embed a microphone in an acoustic cavity in accordance with one aspect of the disclosure;

FIG. 6 is a front, elevational view of one embodiment of the main unit of the electronic prenatal heart listener of FIG. 1 with a number of user interface control elements arranged in accordance with one aspect of the disclosure;

FIG. 7 is a cross-sectional view of the main unit of FIG. 6 taken along lines 7-7 to depict multiple audio outputs in accordance with several aspects of the disclosure;

FIG. 8 is a schematic representation of an exemplary main or base unit of the electronic prenatal heart listener of FIG. 1 to depict control circuitry and other components thereof for capturing, processing, storing, and reproducing heartbeat sounds in accordance with several aspects of the disclosure;

FIG. 9 is an exploded, perspective view of another embodiment of the main unit of the electronic prenatal heart listener of FIG. 1 with front and back housing shells in accordance with one aspect of the disclosure;

FIG. 10 is a perspective view of another embodiment of the auxiliary unit of the electronic prenatal heart listener of FIG. 1 with a dome-shaped body and a semi-spherical acoustic or sound collection cavity in accordance with one aspect of the disclosure; and

FIG. 11 is a plot showing data exhibiting an improvement in fetal heartbeat sound capture using the exemplary prenatal heart listener of FIG. 1.

### DETAILED DESCRIPTION OF THE DISCLOSURE

This disclosure is generally directed to electronic devices and methods for capturing and listening to fetal sounds generated in the womb, including prenatal heartbeat sounds. The disclosed devices provide a reproduction of the captured sounds with improved accuracy and quality, while providing a number of features and functions to facilitate the reproduction. These improvements, features, and functions are provided by the disclosed devices despite being handheld and otherwise convenient for a non-medical personnel user. To these ends, the disclosed devices generally include electronic and stethoscopic instruments and other components distributed over two discrete handheld units, a main (or base) unit and an auxiliary unit. The auxiliary handheld unit is generally dedicated to presenting an acoustic cavity or chamber in which the low-frequency sounds of a fetal heartbeat (and/or other in utero sounds) are collected and captured. The auxiliary handheld unit is generally cup-shaped to define the acoustic cavity and sized to optimize the sound capturing capability of the stethoscope. The auxiliary unit may have a shell body to maximize the size of the interface for a given overall size (e.g., the size of the body of the unit). The auxiliary unit may also have a handle projecting from the body so that the unit is conveniently portable and handheld despite its maximized size.

Several aspects of the disclosed devices involve or relate to the separation and distribution of the stethoscopic components over the two discrete units. Generally speaking, distributing the components over two handheld units (rather than a single composite unit) leads to improved acoustic performance (e.g., frequency response characteristics) as well as user convenience. Multiple units provide convenience in increased freedom of movement and placement. Moreover, a main or base unit may be dedicated to user interface, sound processing, and other control components, while an auxiliary unit may be dedicated to sound capturing components. In this way, the shape, size, and other characteristics of the sound capture unit can be optimized for acoustic performance. That is, allocating all non-sound capture components to the separate main unit (which, in turn, may then be dedicated to sound processing and other control components) provides another way of maximizing the size of the acoustic cavity for a given size of the auxiliary unit. Separation also supports the spacing of the units, which helps to avoid the proximity between components that could otherwise lead to noise interference or other complicating factors for the sound capture. For example, for those embodiments with an on-board speaker for reproducing the captured sounds, the speaker is mounted on, or housed in, the main unit to space the speaker from the microphone or other sound transducer in the auxiliary unit. Allowing a user to avoid the close proximity of the speaker and microphone can help avoid feedback or other interference. Still other aspects of the disclosure involve the distribution and organization of user interface controls, features, and other elements on different sides, panels, or other portions of the main unit depending on whether the elements are directed to heartbeat listening, playback, or other operational modes or features.

Other aspects of the disclosed devices are directed to sound processing techniques, including digital signal processing (DSP) techniques to identify and amplify the sounds of interest, while filtering out unwanted sounds. Sound quality can be improved, for instance, via the implementation of the DSP techniques in conjunction with the improved sound capture features disclosed herein. For example, a housing of a main or base unit of the disclosed devices may enclose a controller or control circuit that includes an audio processor (e.g., a DSP chip) or other processing circuit to implement DSP and other techniques for processing the incoming sound data.

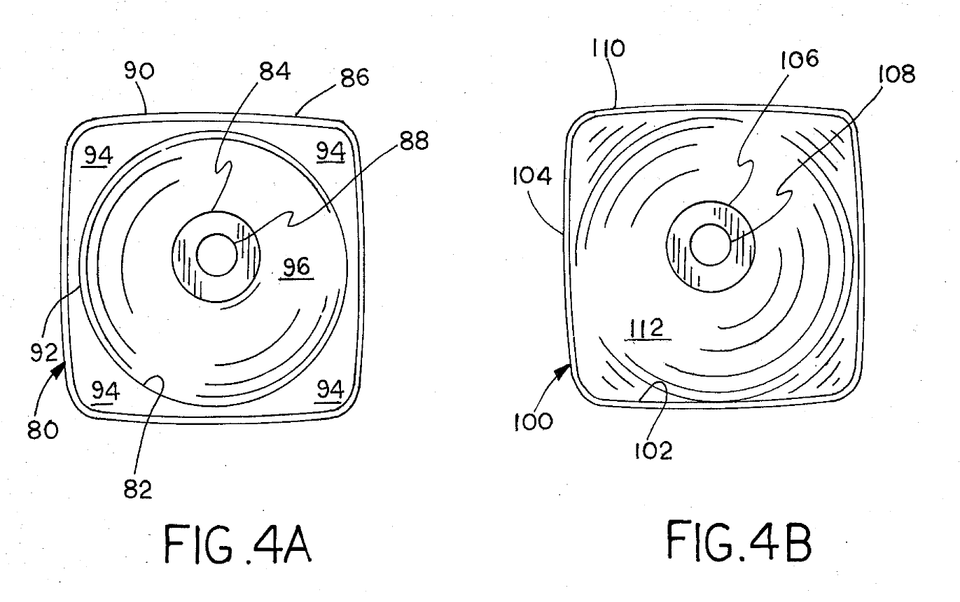

Turning now to the drawing figures, FIG. 1 shows an exemplary prenatal listener device indicated generally at 20 that includes a main or base unit 22 tethered or otherwise communicatively coupled to an auxiliary unit 24 dedicated to sound collection and capture. To that end, the auxiliary unit 24 has a cup-shaped body 26 that defines an acoustic cavity 28 for a microphone (see, e.g., FIG. 4) mounted therein. As described below, the microphone and other aspects of the auxiliary unit 24 are generally configured to capture the low-frequency heartbeat sounds collected by or in the acoustic cavity 28. In this example, the shape of the acoustic cavity 28 generally reflects the cup-shaped nature of the body 26, but this need not be the case. In such cases, however, the body 26 has a shell-like structure or frame with minimal, if any, bulk structure. Minimizing the bulk in the auxiliary unit 24 allows certain dimensions of the acoustic cavity 28 to be maximized while minimizing the overall size of the auxiliary unit 24. For example, maximizing the diameter of an opening 29 of the acoustic cavity 28 can help capture more low-frequency sounds for a given overall size of the auxiliary unit 24.

The auxiliary unit 24 may have a handle 30 to help a user maneuver and position the auxiliary unit 24. Generally speaking, a user can grasp the handle 30 to place the auxiliary unit 24 in contact with the skin of the abdomen to facilitate a monitoring session. In this example, the handle includes a stem or neck 32 that projects upward from a top side of the body 26. The stem 32 supports a knob 34 at a height above the body 26. The components of the handle 30 may be integrally formed with the body 26 in, for example, a one-piece, molded configuration. In this case, the one-piece mold is configured such that the knob 34 necks down or narrows in the area of the stem 32 before the mold flares out to form the body 26. In these and other cases, the handle 30, the knob 34, and/or other components of the auxiliary unit 24 may be formed from or composed of soft rubber materials (or other non-hard materials), which may help to dampen, cancel, or otherwise prevent external noise from the user's hand as it grasps the knob 34.

The construction, shape, and characteristics of the handle 30, the stem 32, and the knob 34 may vary considerably from the example shown, but generally provide a way for a user to grasp the auxiliary unit 24 securely. In this way, the handle 30 may be helpful during a monitoring session, insofar as the handle 30 can be used to steady the auxiliary unit 24 and maintain a good engagement or seal with the abdomen. The handle 30 may also be helpful in maneuvering the auxiliary unit 24 before and after the monitoring session. In the example shown, the knob 34 includes an elongated ridge or rail 36 that runs along a central axis of the handle 30. An end 38 of the ridge 36 or, more generally, the knob 34, may provide a connection interface for a cord 38 that communicatively connects the main unit 22 and the auxiliary 24.

The cord 38 tethers the units 22, 24 to maintain a connection for the signals traveling from the microphone, and also allows the units 22, 24 to be spaced from one another. The spacing may provide several advantages despite the potential for decreased portability arising from having two units rather than a single-unit device. First, the spacing of the two units may generally be useful for convenient positioning of the auxiliary unit 24. That is, the cord 38 allows the auxiliary unit 24 to be spaced from the main unit 22 of the device a desired distance. A considerable amount of distance may be useful in situations where an individual other than the expectant mother is listening to the heartbeat sounds. As described further below, the spacing of the units 22, 24 may also be useful for avoiding feedback or other interference with the sound capture functionality. That is, the spacing and separation leads to improved sound isolation, thereby avoiding interference or noise arising from other, extraneous sounds. In these ways, the cord 38, together with the separate and dedicated nature of the auxiliary unit 24, provide the user with much more freedom of placement for advantages in both convenience and performance.

The separate and dedicated nature of the auxiliary unit 24 also improves sound capture performance in other ways. Because the auxiliary unit is dedicated to sound capture, the shape, size, and other characteristics of the auxiliary unit may be optimized for sound capture functionality. The shape of the acoustic cavity 28 may be optimized or otherwise selected for the low-frequency heartbeat sounds. For instance, the acoustic cavity 28 may have a spherical, parabolic, or other rounded shape when viewed from a vertical or longitudinal (i.e., from top to bottom) cross-section. To this end, the body 26 may also be or include a spherically shaped cup or cone structure. Notwithstanding the foregoing, neither the body 26 nor the acoustic cavity 28 needs to be spherically shaped, as other examples may have a differently shaped sound capture form or structure. For example, one or both of the body 26 and the acoustic cavity 28 may be cone-shaped such that the vertical cross-section is linear. Alternatively, one or both of the body 26 and the acoustic cavity may be bell-shaped, in which case the opening may be flared. The terms “cup,” “bowl,” “cup-shaped,” or “bowl-shaped” are used herein in a general sense to include each of these alternatives, including as well any combinations or other variations of the foregoing shapes. For example, an otherwise spherically shaped cavity may include a flattened top, cover or other portion to, for instance, accommodate the microphone. These and other partially spherical cavities (e.g., hemi-spherical) may be referred to herein as “semi-spherical.”

Other characteristics of the acoustic cavity 28 and, more generally, the auxiliary unit 24, are generally configured to capture the low-frequency sounds of the womb, including the fetal heartbeat. For instance, and as described further below, the size of the acoustic cavity 28 may be optimized for capturing the low frequency heartbeat sounds. The acoustic cavity may also be configured to isolate the desired sounds, thereby avoiding any extraneous sounds. For example, the cavity may be configured with a larger opening to present a greater inlet diameter for capturing sounds. The material(s) and texture of the cavity walls and other surfaces may also be selected in the interest of capturing the low-frequency sounds and isolating extraneous sounds. For instance, in some cases, the interior, side walls defining the cavity may be formed from or otherwise include Acrylonitrile butadiene styrene (ABS), while the top, cover or ceiling of the cavity may be formed from or otherwise include ABS or a thermoplastic elastomer (TPE) material. ABS and other generally dense materials may be used as an inexpensive way to transmit sound effectively to the microphone in the cavity. On the other hand, TPE and other generally soft materials may be used as a convenient way to insulate the microphone from the noise of the user's hand. That is, sounds do not easily transmit through TPE and other soft materials and, thus, the noise from a user holding the auxiliary unit fails to be picked up by the microphone.

With continued reference to FIG. 1, the main unit 22 is generally directed to processing and reproducing the sounds captured by the microphone and, more generally, the auxiliary unit 24. The main unit 22 includes a housing 40 to enclose a number of internal electronic components configured to provide the processing and reproduction functionality. The housing 40 in this example is generally box-shaped, with a front face 42, a top face 44, a bottom face 46, a back face (not shown), and a pair of lateral side faces 48. Each of these faces may have one or more input, output, or other user interface ports or other elements to control and support the functionality of the device 20. In this example, the front face 42 has an input interface panel 50 and a visual output indicator 52. The input interface panel 50 includes a set of four pushbuttons 54, while the output indicator 52 may include a light bar 56 composed of a set of LEDs or other lights to provide status indications and other information based on, for example, the varying activation, intensity, color, etc. of the lights. For example, the light bar 56 may be configured as a soundbar in which the lights operate in series with the number of activated lights being indicative of the volume or intensity of the captured sounds. In this exemplary case, the light bar 56 is seated within a chrome bezel 57. The exemplary main unit 22 also includes a control dial or wheel 58 on one of the side faces 48 to allow user to scroll or otherwise select between various operational settings, modes, or volume levels for the reproduced heartbeat sounds. The dial 58 may also be used as a power ON/OFF switch via either lateral displacement (e.g., an inward push) or rotation past a threshold. In other embodiments, one or more additional control dials or wheels may be included so that the volume and other control settings may be adjusted or selected independently. More generally, the elements, arrangement, and other characteristics of the input interface panel 50 may vary considerably from the example shown. For instance, one or more displays or other visual indicators may illuminate or otherwise display a number of icons to the user to, for instance, identify the operational mode or provide other information.

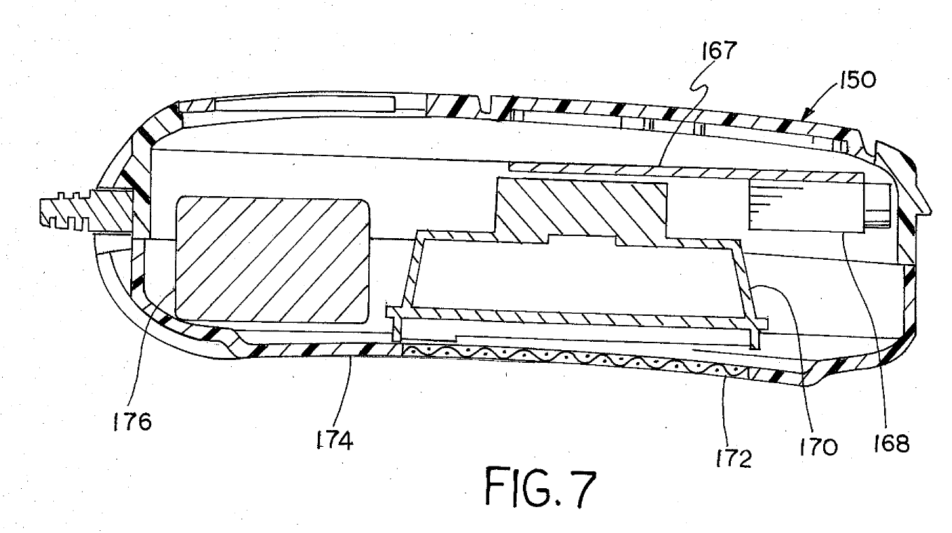

The main unit 22 generally includes one or more audio outputs to reproduce the captured heartbeat sounds. In this example, the top face 44 of the main unit 22 includes a pair of audio output line jacks (FIG. 7) to accommodate a variety of external speakers, such as headphones. In this way, more than one individual may listen to the reproduction of the captured sounds. As shown in FIG. 1, two individuals may listen to the sounds via respective headphone sets 60A and 60B attached via the line jacks. As described further below, the main unit 22 includes another audio output in the form of a speaker (not shown) mounted within the housing 40 (see, e.g., FIG. 7). The speaker advantageously supports a number of listeners without the restrictions of cords, headphone sets, or other connections to the main unit 22. With that advantage, the speaker also presents one or more challenges met by various aspects of the disclosed devices described below. For example, the spacing and separation of the units 22, 24 helps to avoid feedback or other interference between the speaker and the microphone.

In accordance with one aspect of the disclosure, the main unit 22 is also configured to be handheld. While the shape, size, and other physical characteristics of the main unit 22 may vary from the example shown, the handheld nature of the main unit 22 provides a number of advantages. For example, as a handheld unit, the main unit 22 can easily be relocated to allow the audio output(s) to be re-positioned for the convenience of the user(s), or to facilitate one or more non-listening operational modes, as described below.

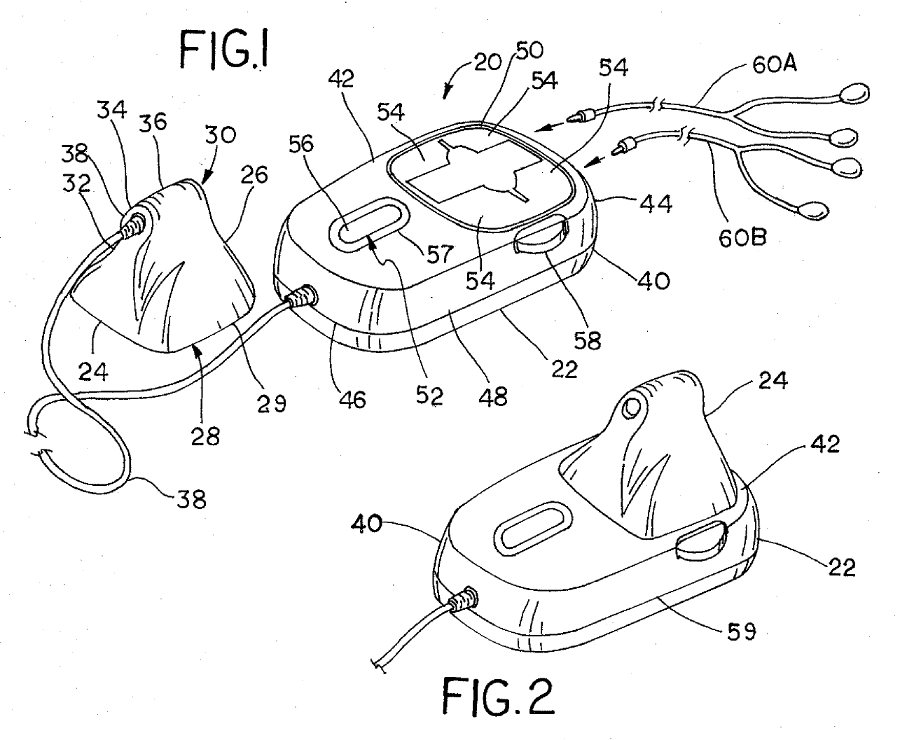

With reference now to FIG. 2, the device 20 is shown in a storage configuration in which the auxiliary unit 24 is removably attached to the main unit 22. The storage configuration allows the units 22, 24 to be secured together as a single, integrated item to avoid loss or damage to the units 22, 24 while not in use as an instrument for monitoring or recording heartbeat sounds. In this example, the auxiliary unit 24 may snap into a groove (not shown) or other depression in the front face 42 of the housing 40 (see, e.g., FIG. 6). The attachment of the units 22, 24 need not be a snap-fit or press-fit connection, and instead may utilize a variety of different latches or other fasteners (e.g., hook and loop). Furthermore, the positioning and orientation of the auxiliary unit 24 in the depicted storage configuration is also exemplary nature, in the sense that the units may be attached on a different face (e.g., the back face of the housing 40) and in a variety of orientations (e.g., the auxiliary unit 24 need not be oriented with the opening 29 engaging the main unit 22). However, the orientation shown allows the opening 29 to accept or receive portions of the main unit 22 to secure the connection. For example, if the auxiliary unit 24 engages a groove in the front face 42, any portion of the pushbuttons 54 (FIG. 1) or other component of the input interface 50 can extend into the acoustic cavity 28 (FIG. 1) via the opening 29 once the units are snapped together. Such positioning within the cavity 28 may help stabilize the engagement and protect the user interface elements from damage. The storage configuration may also include a cord management system or feature in which the cord 38 engages a groove (not shown) in the housing 40. For example, the groove may be formed along an interface 59 between component parts of the housing, an example of which is described below.

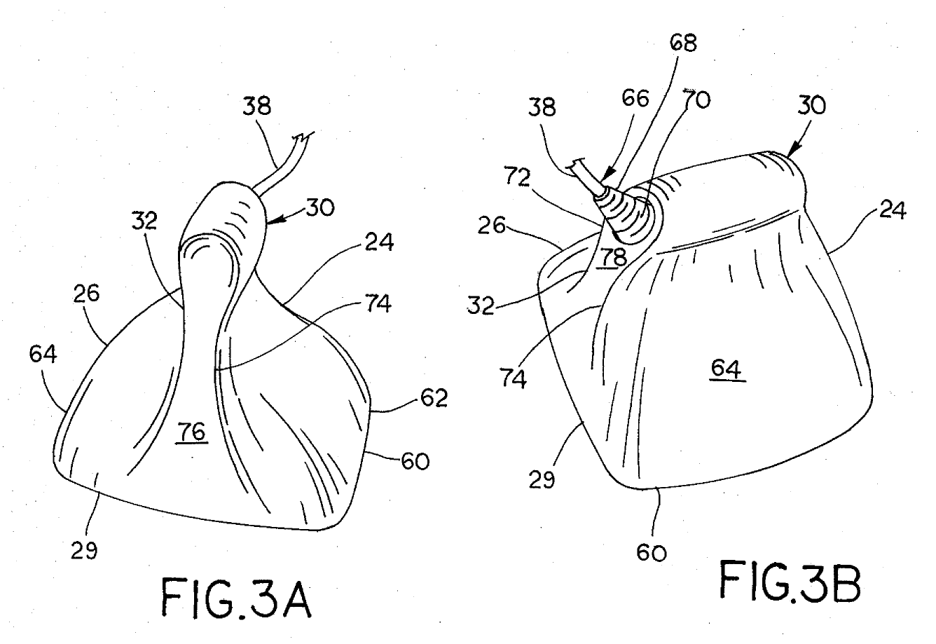

FIGS. 3A and 3B depict the exemplary auxiliary unit 22 to show the body 26 in greater detail. The body 26 generally tapers inward from a rim or skirt 60 that defines the opening 29, eventually forming the neck or stem 32 of the handle 30. In this case, the rim 60 has a generally square shape when viewed from above or below. The square shape of the rim 60 simplifies the tapering of the body, insofar as the length of lateral sides 62, 64 generally matches the length of the elongated handle 30. Other shapes may also be used, including, for instance, a circular shape to accommodate a semi-spherical acoustic cavity. The rim 60 may have a varying thickness to accommodate non-circular shapes of the body 26. One advantage of having a square- or rectangular-shaped rim involves the engagement of the units 22, 24 in the storage configuration. A square or rectangular shaped rim may be more compatible with the shape of the user interface elements on the main unit 22.

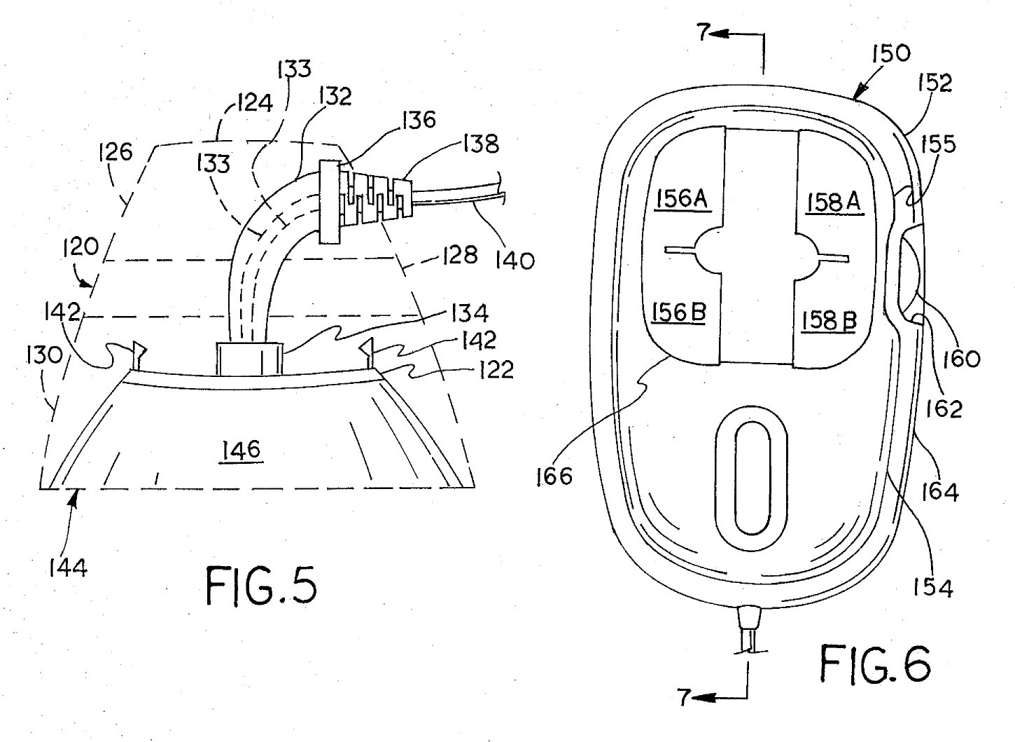

The elongated nature of the exemplary handle 30 generally facilitates the connection of the cable 38 to the auxiliary unit 24. In this example, an end 66 of the cable 38 has a stress-relief cone 68 that has a number of grooves or slots to allow and regulate a limited amount of deflection in the cable 38. In this way, the cable 38 can bend during adjustments of the position of the auxiliary unit 24 without fatigue or other undesirable wear. The stress-relief cone 68 extends out of an opening in one of two longitudinal end faces 72 of the handle 30, as best shown in FIG. 3B. Behind the face 72, the cable 38 is connected to wiring (FIG. 5) that leads to the microphone. This connection path through the end face 72 avoids interfering with a user's grasp of the handle 30 and, more generally, the auxiliary unit 24. The connection path also minimizes interference with the sound capture functionality of the auxiliary unit 24, insofar as the cable 38 enters the auxiliary unit 24 at a point spaced from the components of the auxiliary unit 24 collecting the heartbeat sounds.

The body 26 may have a molded construction to achieve and maintain satisfactory acoustic performance, as well as to simplify assembly and minimize the part count. For instance, the molded construction may be one-piece or two-piece, and may be formed with a hole to receive the microphone. A two-piece construction may be used, for instance, for ease in manufacturing or assembly. In this example, an outer surface of the body 26 is provided or formed by a one-piece cap 74 that defines the shape and exterior surface of the handle 30, the neck 32, the lateral sides 62, 64, a front side 76 (FIG. 3A), and a rear side 78 (FIG. 3B). The cap 74 extends the entire height of the unit 24, i.e., from the handle 30 to the rim 60, and may also include or define part or all of the inner surfaces or walls (i.e., within the acoustic cavity). In this example, the cap 74 has an inward facing surface that defines the acoustic cavity. Further details regarding the formation of the outer and inner surfaces of molded constructions of the auxiliary unit 24 are provided in connection with the exemplary embodiments shown in FIGS. 4A, 4B, and 5.

FIG. 4A shows an example of an auxiliary unit 80 with a semi-spherical acoustic cavity 82 formed from an insert 84 received within an outer cap or shell 86. The insert 84 acts as a generally flat cover or ceiling within the cavity 82 and includes a receptacle for a microphone 88. The insert 84 may alternatively be curved to match the curvature of the outer cap 86, in which case the acoustic cavity 82 may approach or form a hemisphere. More generally, the insert 84 may be secured to the shell 86 via a variety of fastener types, including those that provide a smooth interface between the insert 84 and the shell 86. In this case, the outer shell 86 defines an outer rim 90 and an inner rim 92. A generally flat shelf 94 spaces the inner rim 92 from the outer rim 90, primarily in corner regions where the transition from the square-shaped rim 90 to the round inner rim 92 is greatest. The shelf 94 is disposed at a height relative to the outer rim 90 to allow the outer rim 90 to engage the front face of the main unit, as shown in FIG. 2, while providing clearance for buttons and other user interface elements. From that height, a dome-shaped wall 96 defines the cavity 82, curving upward from the inner rim 92 to reach the insert 84. In this example, the curvature of the wall 96 and, thus, the cavity 82 is semi-spherical, such that the shape of the wall 96 is a circular arc when viewed in any vertical cross-section.

A variety of condenser microphones or other acoustic transducers or sensors may be used in the auxiliary unit 80. In some cases, the microphone 88 is an electret microphone, a configuration that avoids having to provide a separate power supply in the auxiliary unit 80. Other suitable examples include dynamic microphones having a diaphragm or a piezoelectric (e.g., ceramic) disc to capture the heartbeat sounds. Thus, the disclosed devices and techniques are compatible with a variety of different types of microphones. In each case, however, the microphone-based technique for capturing the heartbeat sounds is non-invasive. Some of the microphone constructions or types, such as a piezoelectric buzzer, may be more effective at filtering sounds from outside the womb than others, but less effective at detecting and gathering sounds from the womb. Notwithstanding the foregoing, the configuration and construction of the microphone 88 in the auxiliary unit 80 may vary considerably, insofar as the disclosed devices and techniques are not limited to any particular microphone type. The term “microphone” is used herein in a broad sense to include each of the aforementioned types of acoustic transducers or sensors, and should not be limited to a particular type of condenser or other microphone.

FIG. 4B shows another exemplary auxiliary unit 100 with a non-spherical acoustic cavity 102 that extends upward from an outer rim 104 to reach an insert 106. The insert 106 may act as a cover or ceiling within the cavity 102, include a receptacle for a microphone 108, and generally be configured in any one of the manners described above in connection with the embodiment of FIG. 4A. In contrast to the dome-shaped wall 96 of that embodiment, an outer cap or shell 110 has an inner surface 112 that generally tracks the shape of the outer surface from the outer rim 104 upward (see, e.g., FIGS. 3A and 3B). In this way, the non-spherical nature of the cavity 102 around and beneath the insert 106 is established.

One way in which the above-described inserts may be secured within an outer cap of the auxiliary unit is depicted schematically in the embodiment of FIG. 5. In this example, an auxiliary unit 120 of two-piece, molded construction has an insert 122 that engages an outer cap 124 shown in phantom for ease in illustration. A side view of the auxiliary unit 120 is depicted, such that the width of a handle section 126, a neck section 128, and a base section 130 (truncated for case in illustration) of the outer cap 124 are shown. Each section of the outer cap 124 may be solid or hollow to any desired extent. A conduit path 132 runs from the handle section 126 to carry wiring 133 to a microphone 134 mounted within a receptacle (not shown) in the insert 122. Within the handle section 126, the conduit path 132 ends at a coupler 136 to which a stress-relief tip 138 is attached. The wires 133 pass through the coupler 136 to continue on through cabling 140 that connects the main and auxiliary units.

The insert 122 may include a number of clips 142 or other fasteners for engagement with the outer cap 124. In this example, the clips 142 are positioned opposite one another, extending upward from a perimeter of the insert 122. Each clip 142 may form part of a cantilever or other snap-fit (or press-fit) connection, in which case the outer cap 124 has corresponding receptacles (not shown) for engagement with the clip 142. During assembly, the clips 142 are deflected radially outward until snapping into place within respective receptacles. The assembly of the insert 122 and the outer cap 124 may also secure the microphone 134 in position by trapping the microphone 134 between the two components. The nature of these connections may vary considerably, as desired. In some cases, for instance, the microphone 134 may be secured only to the insert 122 through a pressure-fit connection or other fastening technique.

FIG. 5 also shows a portion of an acoustic cavity 144 to provide another example of a non-spherical cavity. Like the above-described cavities, the cavity 144 is symmetrical about the microphone 134 and, as such, is defined by an inner wall 146 that revolves around the microphone 134. In vertical cross-section, the inner wall 146 is curved, but less dramatically than the circular arcs of the semi-spherical cavities described above. In some cases, the curvature may be parabolic so that the microphone 134 can be positioned as in a parabolic microphone configuration. Although the exterior of the wall 146 is shown in the schematic depiction of FIG. 5, the wall 146 may be formed as a part of a solid mold of the cap 124. Alternatively, when the cap 124 is hollow, the mold that leads to the cap 124 defines the wall 146 as an interior component linked to an outer shell at the rim (see, e.g., FIGS. 4A, 4B) of the body.

With reference now to FIG. 6, another exemplary base or main unit 150 is shown to exhibit several other aspects of the disclosure. Like the above-described main unit 22 (FIG. 1), the main unit 150 includes a handheld housing 152 to enclose a controller or control circuitry, while also presenting a number of user interface elements. Generally speaking, the main unit 150 is configured to maximize ease-of-use with user-friendly operational features for controlling and utilizing the sound capturing functionality. In this example, the housing 152 includes a front side 154 with a panel 155 having any number of user interface control buttons and other elements.

In some embodiments, the user interface elements are disposed, organized or arranged based on functionality. Those elements directed to the heartbeat capture and collection are arranged in one group. Other elements, such as those directed to sound playback and other output, are then arranged in another group. Sound playback and other output may be utilized for soothing or communicating with the baby in the womb. While the depicted example has been simplified for ease in illustration, the front panel 154 organizes and separates these two groups with an arrangement of two pairs of user interface buttons 156A, 156B and 15SA, 158B. The first pair of user interface buttons 156A, 156B are on one side of the panel 154, and may be directed to capture and collection functions, such as on/off power, volume control, and headphone jacks. On the other or opposite side of the panel 154, the second pair of user interface buttons 158A, 158B may be directed to activating or enabling one or more listening functions, including, for instance, sound playback from either internal or external music sources. To these ends, the housing 152 may include an input jack (not shown) for a connection with an external source of sound or music (e.g., a portable music player).

In some cases, one or more of the buttons 156A, 156B, 158A, 158B maybe directed to selecting an operational mode for the device by, for instance, toggling between a plurality of operational modes. Different modes may be directed to different sound capture contexts, or whether the microphone is capturing sound at all. For example, one operational mode may be dedicated to capturing heartbeat sounds. To that end, amplification, volume, and other operational settings may be preset or configured with default values suitable for capturing and listening to the heartbeat sounds. Another operational mode may be available for capturing and playback of sounds spoken by a parent to be focused and directed to the womb by a speaker (FIG. 7) mounted in the unit 150. Still further operational modes may be directed to playback of music or other sounds not collected by an auxiliary unit microphone (see, e.g., FIG. 5), but rather provided by an internal or external audio source.

The user interface elements of the main unit 150 are not limited to those disposed or mounted on the front panel 154. For example, the main unit 150 may include a scroll wheel or dial 160 projecting from a notch 162 in a lateral side or edge 164 of the housing 152. The scroll wheel 160 may be directed to volume changes or a variety of other adjustments. In some cases, the control variable to which the wheel 160 is directed may be selected by a user interface button, such that the wheel 160 can control any one of a plurality of features depending on a current selection, operational mode, or other context. The layout, number, size, shape, and other characteristics of the user interface elements of the main unit 150 may vary considerably from those depicted in FIG. 6, as desired. For instance, the user interface elements 156A, 156B, 158A, 158B may be touch-sensitive pads (or part of a touch-sensitive display screen) rather than push-buttons.

The exemplary embodiment shown in FIG. 6 also depicts another alternative mounting arrangement for connecting an auxiliary unit in the manner shown in FIG. 2. In this case, the auxiliary unit engages an edge 166 of the panel 154 via an interference fit. This connection is in contrast to an engagement of a groove or other depression, as described above in connection with the embodiment of FIGS. 1 and 2. To that end, either the edge 166 or the rim of the auxiliary unit (see, e.g., FIGS. 4A, 4B), or both the edge and the rim, may deflect to form the interference-fit engagement. A variety of other interference-fit, pressure-fit, or snap-fit arrangements may alternatively or additionally be used to establish the storage configuration.

FIG. 7 depicts an exemplary internal arrangement of electronic components within the main unit 150. A controller (not shown) and a number of constituent or other electronic components may be mounted on a circuit board 167, which, in turn, may be mounted beneath or near the user interface panel 154 to accommodate connections therewith. In some cases, the circuit board 167 includes a conventional printed circuit board (PCB) for receiving a number of discrete components, including, for instance, one or more headphone output jacks 168. The output jack 168 may provide one of several audio outputs for the main unit 150. For instance, a speaker 170 may also be mounted within the main unit 150. The speaker 170 may be disposed behind or adjacent to a screen, grating, or other opening 172 that forms part of a rear side 174 of the housing 152. In this way, the opening in the housing accommodates the propagation of the captured sounds from the speaker 170. A number of other components may also be enclosed within the housing 152, including, for instance, one or more battery cells 176.

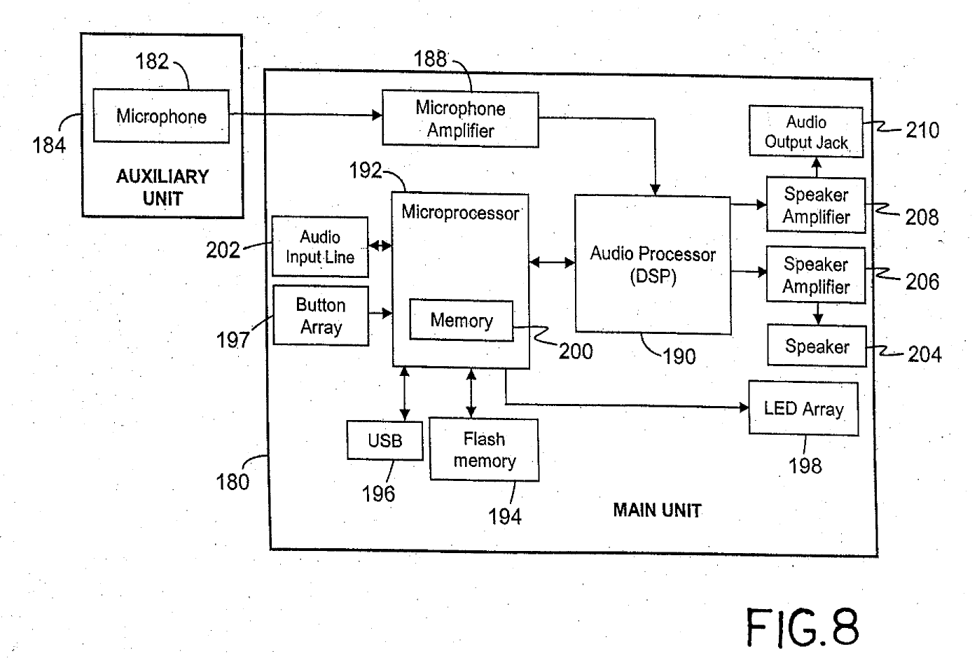

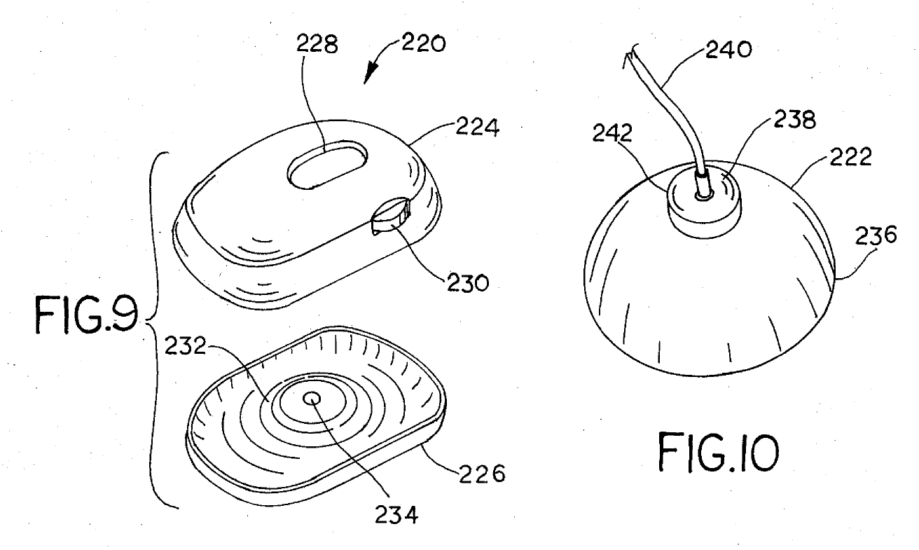

FIG. 8 depicts a schematic representation of the electronic components of an exemplary main or main unit 180. Heartbeat or other sounds are converted to an analog signal by a microphone 182 (or other audio transducer or sensor) of an auxiliary unit 184. The analog signal is then provided to the main unit 180 via a cable 186 or other communication connection, which may be wireless (e.g., IEEE 802.11, Bluetooth, or other standards). The analog signal is then amplified with a microphone amplifier 188. In some cases, the analog signal may then be converted to digital data by a separate (e.g., discrete) analog-to-digital converter (not shown). In this example, the analog representation of the captured sounds is provided to an audio processor 190 that has an integrated analog-to-digital converter.

The audio processor 190 is generally directed to optimizing the reproduction of the fetal heartbeat and other sounds that can be difficult to hear (e.g., low-frequency sounds). To that end, digital signal processing (DSP) techniques and methods may be used to identify and amplify the captured sounds. In this example, the audio processor 190 is configured to perform a number of processing operations in addition to preparing digital representations of the captured audio. For example, the signal processing may generally gather the low-frequency heartbeat sounds (e.g., 20-50 Hz), and move them to a higher frequency range easier for humans to hear. In these and other cases, the signal processing may generally amplify any audible or partially audible sounds. The signal processing may also include or incorporate filtering techniques to remove unwanted sounds. These processing techniques in combination with the above-described characteristics of the auxiliary unit 184 have been found to greatly improve the quality of prenatal heartbeat sounds captured by the disclosed devices.

The main unit 180 may include a general-purpose processor or microprocessor 192 to package the output of the audio processor 190 into data packets suitable for storage in a memory device, such as a flash memory chip 194, or for delivery to an external device via a USB port 196. For example, the microprocessor 192 may configure the data packets to include a variety of other information in addition to the content data, including identification headers, synchronization data, error detection, and other information. Alternatively or additionally, these and other tasks may be handled by the audio processor 190. More generally, any number or configuration of processors or processing elements may be involved in the implementation of these digital signal processing tasks. For example, a single microprocessor may be configured to handle the tasks implemented by both the audio processor 190 and the microprocessor 192 of the example shown.

The processing provided by the audio processor 190 or the microprocessor 192 may involve or implement one or more coding protocols or techniques to support the digital transmission and downstream processing of the captured audio. For example, the audio processor 190 may implement one or more compression, compilation, filtering, packet organization, or other procedures to organize or present the raw data in a more refined or convenient format. The procedures may correspond with data conversions and other techniques established by, and in accordance with, one or more audio data standards or protocols. To those ends, the audio processor 190 may be programmed or otherwise configured to implement any number of sets of codec instructions stored in the form of software, firmware, hardware, or any combination thereof.

A variety of different commercially available integrated circuits may be used as the audio processor 190, including, for example, the programmable multimedia processors from STMicroelectronics Corp (www.st.com), and the audio/video chip having a multimedia processor available from Winbond Electronics Corp. (www.winbond.com) as product no. W99702G. These chips may be configured to include any number of codecs for audio processing in accordance with conventional data protocols (e.g., MP3). In some cases, the audio processor 190 includes more than one processor (or processing element) integrated within a system-on-a-chip architecture with embedded memory and other integrated components.

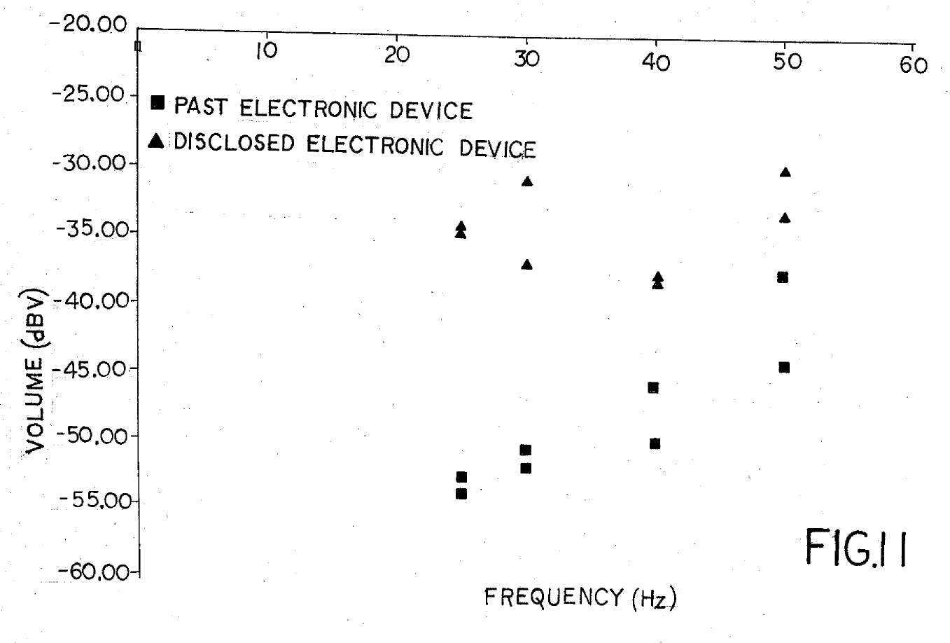

The microprocessor 192 in the example shown in FIG. 10 may include a general-purpose processor directed and configured to control the various operations and functions of the main unit 180. For example, the microprocessor 192 may be programmed to implement tasks related to preparing a digital data stream for USB transmission. To that end, the microprocessor 192 may implement one or more routines directed to arranging the content data prepared by the audio processor 190 into data packets or frames suitable for USB (Universal Serial Bus) transmission via a USB port 196. The microprocessor 192 may also be configured to implement one or more tasks or routines related to controlling or responding to input or output user interface elements. For example, the microprocessor 192 may receive directions from any number of user interface elements, including, for instance, an array of interface control buttons 197. On the output side, an LED array 198 may be provided with control signals developed by the microprocessor 192 to indicate status and other operational conditions. To these ends, the microprocessor 192 may have one or more on-board or embedded memories 200 in which instructions or audio data are stored. The memory 200 need not be fully contained within the microprocessor package as shown in the example of FIG. 10, and may be of any desired size, type, location, number, etc. For example, the memory 200 may be volatile SDRAM or SRAM, non-volatile flash memory, or a user-removable flash memory card.

In some cases, the tasks handled by the audio processor 190 may be shared with, or implemented by, the microprocessor 192 instead. More specifically, the microprocessor 192 may be configured to prepare a digital representation(s) of the captured audio in addition to implementing any one or more coding and conversion techniques to support the digital transmission of the content data. In these cases, the main unit 180 may not need separate processors, and instead may only have a single microcontroller or ASIC to handle commands, instructions, or other information other than the content data. Indeed, either the audio processor 190 or the microprocessor 192 may be programmable to an extent to implement these other tasks involved in support of the capture, processing, and communication of the audio data.

In the example shown, the microprocessor 192 is also configured to receive audio data via an audio input line or jack 202. In this way, an external source of music or other audio may be connected to the main unit 180 for playback via a speaker 204. To that end, the microprocessor 192 may pass the external audio data to the audio processor 190, which then accordingly controls a speaker amplifier 206 dedicated to driving the speaker 204. In other cases, the audio processor 190 may receive the external audio data directly. In these and other cases, the audio processor 190 or the microprocessor 192 may receive the audio data for playback to a fetus via the speaker 204 from an internal memory or other source, such as the flash memory 194 or the memory 200. With the flexibility of a number of different sources or storage locations, the audio data may include soothing sounds of a variety of types (e.g., fixed, variable, etc.), including music, a recording of a parent's voice, or any other desired sound.

In the heartbeat listening mode, the captured audio data may be provided to the audio processor 190 directly from the microphone amplifier 188. The audio processor 190 then implements one or more D SP routines to improve the sound quality before passing the modified audio data to one or both of the speaker amplifier 206 or another speaker amplifier 208 dedicated to driving one or more headphones (FIG. 1) connected to audio output jack(s) 210. A switch (not shown) or other control element may be included to determine whether the speaker amplifier 206 is driven. The switch or other control element may be configured such that the determination is, for instance, based on whether any headphones are plugged into the output jack(s) 210.

The flash memory 194 or other onboard or resident memory is generally directed to recording the sounds captured by the microphone 182. Once captured, the sounds may then be played back at a convenient, later time via the speaker 204 or the headphone jack(s) 210. One or more user interface elements (FIG. 6) may be directed to controlling an operational mode dedicated to the playback of stored audio.

The main unit 180 may include any number of output data ports to export the captured audio data to an external device. The configuration, transmission protocol, and other characteristics of the output data ports may vary considerably from the USB port 196 shown, as desired. In some cases, one or more of the data ports are wireless (e.g., Infrared, IEEE 802.11, Bluetooth, etc.).

FIGS. 9 and 10 depict an alternative main or base unit 220 and an alternative auxiliary unit 222, respectively, each of which are sized and generally configured for handheld use. The main unit 220 includes a housing composed of an upper shell 224 and a lower shell 226. Either shell 224, 226 may be configured to present any number of user interface elements, such as control buttons, display screens, and dials. In this example, the upper shell 224 is shown for ease in illustration with two interface elements, an input/output display panel 228 and a scroll wheel or dial 230. The user interface elements may vary considerably from the examples shown. The lower shell 226 in this example includes a depression 232 sized and configured to receive or act as a speaker (not shown). A connection to the diaphragm (not shown) of the speaker may pass through a central aperture 234 in the depression 232. More generally, the example of FIG. 9 shows that the speaker may be mounted or configured in a variety of ways in addition to the example described above in connection with the embodiment of FIG. 7.

The auxiliary unit 222 shown in FIG. 10 includes a bowl-shaped shell 236, which accordingly forms a bowl-shaped acoustic cavity. In this example, the shape of the bowl and, thus, the acoustic cavity are both semi-spherical. A microphone assembly 238 is centered at a top of the acoustic cavity to capture the sounds collected within the shell 236. A cable 240 connects the microphone assembly 238 to a main unit for delivery of the analog signals representative of the captured sounds. The microphone assembly 238 may be mounted or disposed in the shell 236 such that a ridge 242 projects upward from the shell 236 to an extent that provides space for the microphone. The ridge 242, or an expanded or extended version thereof, may also allow the ridge 242 to act as a handle for a user. Additional material added to the ridge 242 as shown may be added to provide more area for the user to hold.

Several aspects of the above-described auxiliary units lead to a larger acoustic cavity for collection of fetal or other in utero sounds, which can be useful with the low frequencies presented by fetal heartbeat sounds. For example, the discrete nature of the auxiliary unit allows the shape, structure, components, and other characteristics to be optimized for sound collection. In this way, other aspects or components of the listening device do not impede the design or, more specifically, the size of the cavity. Separating the aspects and components of the listening device over the two units allows the auxiliary unit to be dedicated to sound capture features. Other features, like user interface controls, power supply(ies), control circuitry, speakers and speaker connections, memory, etc., to be allocated to the main unit. An auxiliary unit dedicated to sound capture allows the body of the auxiliary unit to be customized for optimal sound collection capability given a certain, overall size. For instance, some embodiments may have an auxiliary unit with a shell body, such that the unit has little to no excess bulk or structure beyond the components directly involved in the sound collection. One exception is the handle, which, in the examples described above, may be an integrated, molded component that facilitates positioning while also providing a convenient, non-obtrusive connection location for the cable. A separate, discrete auxiliary unit dedicated to sound capture also allows the overall device to remain comfortably handheld and, thus, portable. The need to transport and handle two units may be seen as a decrease in handling convenience in some cases, but handling during use may be simpler with two handheld units rather than a single, bulky unit. This advantage may be especially applicable to situations in which the expectant mother is also the user positioning the auxiliary unit and/or listening to the heartbeat sounds. Two, separate units would be more convenient for the mother than a single, bulky unit The handling of two units (when the device is not in use) is also addressed via another aspect of the disclosed devices, i.e., the snap-fit storage arrangement of the auxiliary and main units.

These and other aspects of the auxiliary units described above generally allow the acoustic cavity to be as large as the anatomy can accommodate. In other words, the diameter of the opening and overall volume of the acoustic cavity may be as large as possible given the condition that the rim defining the opening remain in contact with the abdomen. Maintaining a good seal with a larger opening or overall volume can be challenging in light of the curvature of the abdomen during pregnancy. The handles of the auxiliary units described above may become useful for maintaining the good seal or contact. Other operating conditions where the rim fails to engage the abdomen generally fail to collect the heartbeat sounds sufficiently. Thus, the above-described auxiliary units are sized and otherwise configured for full engagement with the abdomen (e.g., around the entire circumference of the opening to the acoustic cavity). Notwithstanding the foregoing, the nature of the disclosed devices do not require contact with the skin of the abdomen. Unlike other monitoring devices (e.g., Doppler devices), the disclosed devices can still capture the fetal or in utero sounds when placed on top of clothing covering the skin.

Maximizing the overall volume of the acoustic cavity for a given diameter opening may then be accomplished given the features and aspects of the auxiliary unit described above. In some cases (e.g., semi-spherical cavity examples), the diameter of the opening generally establishes the overall volume, but, in any case, the above-described aspects of the auxiliary unit may still be useful for minimizing the overall size of the auxiliary unit. In some examples, the opening into the acoustic cavity has a diameter (or effective diameter in non-circular configurations) in a range from about 40 mm to about 60 mm. In other cases, the diameter of the opening may be as large as possible while accommodating the interface with the expectant mother.

FIG. 11 shows a plot of test result data reflecting a significant improvement in sound capture using the device of FIG. 1. Generally speaking, the devices constructed as described above, including, for example, the half-sphere shape of the acoustic cavity, have been found to provide more accurate results. More specifically, FIG. 11 depicts a comparison of the frequency response of an existing device having a bell (or cone) with a device having the cup-shaped auxiliary unit described and shown in connection with FIG. 1. The data for the device with the cup-shaped auxiliary unit is represented by triangles, which collectively reflect a set of consistently louder sounds, especially at the lower frequencies of interest.

Described above are a number of electronic prenatal heart listeners having an auxiliary unit dedicated to capturing sounds in the womb. The auxiliary unit has a cup-shaped body that forms a cup-, cone-, or bell-shaped acoustic cavity (or chamber) configured to capture low frequency sounds of a fetal heartbeat in accordance with one or more aspects of the disclosure. In some cases, one or more speakers may be disposed within the sound capture cone or bell in addition to the microphone directed to capturing the sounds. In that way, the auxiliary unit may be used for sound playback in the operational mode described above involving reproduction of audio data from an external source.

A number of devices and methods are described above for capturing low-frequency sounds produced within the womb. The devices and methods generally involve a pair of discrete units configured to capture the low-frequency sounds. These and other aspects of the disclosed devices and methods present inexpensive and noninvasive solutions for prenatal listening or monitoring. Each device has a limited number of parts to reduce construction complexity and provide a more robust product. The shape of the sound capture unit of the disclosed devices may be combined with electronic sound processing techniques (e.g., DSP techniques) and user-friendly electronic control elements to provide a better, more comprehensive product for parents seeking a solution for listening to prenatal sounds at home or away from the doctor's office.

Although certain devices have been described herein in accordance with the teachings of the present disclosure, the scope of coverage of this patent is not limited thereto. On the contrary, this patent covers all embodiments of the teachings of the disclosure that fairly fall within the scope of permissible equivalents.

What is claimed is:

1. A prenatal listening device comprising:
a first handheld unit including a cup-shaped body that defines an acoustic cavity, the first handheld unit further including a microphone mounted to the body in the acoustic cavity to capture fetal sounds collected by the acoustic cavity; and
a second handheld unit spaced from and communicatively coupled to the first handheld unit, the second handheld unit including a controller to process signals from the microphone indicative of the captured fetal sounds, a housing to enclose the controller, and an audio output to reproduce the captured fetal sounds.

2. The prenatal listening device of claim 1, wherein the audio output comprises a speaker, and wherein the housing comprises an opening adjacent the speaker to accommodate propagation of the captured sounds from the speaker.

3. The prenatal listening device of claim 1, wherein the acoustic cavity is semi-spherical.

4. The prenatal listening device of claim 1, wherein the first handheld unit further includes a handle extending from the cup-shaped body.

5. The prenatal listening device of claim 4, further comprising a cord that connects the first and second handheld units, wherein the handle has a connection face for the cord.

6. The prenatal listening device of claim 1, wherein the cup-shaped body of the first handheld unit includes a rim, and wherein the second handheld unit includes a face configured for engagement by the rim to form a storage configuration of the first and second handheld units.

7. The prenatal listening device of claim 1, wherein the controller includes an audio processor configured to implement digital signal processing techniques on the captured fetal sounds.

8. The prenatal listening device of claim 1, wherein the cup-shaped body is a shell having an inner face that defines the acoustic cavity.

9. The prenatal listening device of claim 1, wherein the controller is configured to amplify the captured fetal sounds.

10. The prenatal listening device of claim 1, wherein the cup-shaped body of the first handheld unit includes a rim configured to define an opening of the acoustic cavity without requiring skin contact for capture of the fetal sounds.

11. The prenatal listening device of claim 1, wherein the controller is configured to record the captured fetal sounds.

12. The prenatal listening device of claim 1, wherein the controller is configured to reproduce stored sounds for playback via the audio output to a fetus.

13. A prenatal listening device comprising:
a first handheld unit including a cup-shaped body that defines an acoustic cavity, the first handheld unit further including a microphone mounted to the body in the acoustic cavity to capture fetal sounds collected by the acoustic cavity; and
a second handheld unit spaced from and communicatively coupled to the first handheld unit, the second handheld unit including a controller to process signals from the microphone indicative of the captured fetal sounds, a housing to enclose the controller, and a speaker to reproduce the captured fetal sounds.

14. The prenatal listening device of claim 13, wherein the acoustic cavity is semi-spherical.

15. The prenatal listening device of claim 13, wherein the first handheld unit further includes a handle extending from the cup-shaped body.

16. The prenatal listening device of claim 15, further comprising a cord that connects the first and second handheld units, wherein the handle has a connection face for the cord.

17. The prenatal listening device of claim 13, wherein the cup-shaped body of the first handheld unit includes a rim, and wherein the second handheld unit includes a face configured for engagement by the rim to form a storage configuration of the first and second handheld units.

18. The prenatal listening device of claim 13, wherein the controller includes an audio processor configured to implement digital signal processing techniques on the captured fetal sounds.

19. The prenatal listening device of claim 13, wherein the cup-shaped body is a shell having an inner face that defines the acoustic cavity.
